(19) United States
(12) Patent Application Publication
(10) Pub. No.: US 2009/0305247 A1
(43) Pub. Date: Dec. 10, 2009

(54) NANOPARTICLE AND METHODS THEREFOR

(76) Inventor: Zhiqiang Gao, Singapore (SG)

Correspondence Address: KLARQUIST SPARKMAN, LLP, 121 SW SALMON STREET, SUITE 1600, PORTLAND, OR, 97204, US

(21) Appl. No.: 12/095,500

(22) PCT Filed: Nov. 30, 2006
(86) PCT No.: PCT/SG2006/000368

§ 371 Date: May 29, 2008

Related U.S. Application Data
(60) Provisional application No. 60/740,676, filed on Nov. 30, 2005.

(51) Int. Cl. C12Q 1/68

(52) U.S. Cl. 435/6; 977/920

(57) ABSTRACT

There is provided an electroactive nanoparticle, which may be used as a label in electrochemical detection assays. The nanoparticle comprises a transition metal oxide and a capping agent, the capping agent comprising a ligand group and a functional group. The capping agent is coordinated to a transition metal centre in the transition metal oxide via the ligand group. Also provided are methods relating to preparation of the nanoparticle and detection of an analyte molecule in a sample using electrochemical methods.

### CROSS-REFERENCE TO RELATED APPLICATION

This application claims benefit and priority from U.S. provisional patent application No. 60/740,676, filed on Nov. 30, 2005, the contents of which are incorporated herein by reference.

### FIELD OF THE INVENTION

The present invention relates generally to nanoparticles and to electrochemical detection methods using such nanoparticles.

### BACKGROUND OF THE INVENTION

Detection of various types of analyte molecules in a sample is commonly used in a wide range of fields, including clinical, environmental, agricultural and biochemical fields. Currently, various techniques are available for the detection and quantification of analyte molecules in a sample, including immunoassays for the detection of proteins, PCR methods for the detection of nucleic acid molecules and blotting techniques for the detection of smaller oligonucleotides.

Electrochemical assays have also been developed as methods for detection of analyte molecules in a sample. Such assays provide ease of detecting electrochemically active molecules and eliminate the need for specialized and complicated detection devices. Electrodes used in detection of the electrochemically active molecules can be miniaturized for inclusion in portable devices for point-of-care and field uses. Furthermore, the electrodes can be easily arranged into microarray platforms for multiplexing applications.

There exists a need for a method for detecting analyte molecules in a sample, which method is sensitive and simple to use. There is a particular need for such a method that is capable of easily and efficiently detecting and/or quantifying short nucleic acid molecules.

### SUMMARY OF THE INVENTION

In one aspect, there is provided a nanoparticle comprising a transition metal oxide and a capping agent, the capping agent comprising a ligand group and a functional group, the capping agent coordinated to a transition metal centre in the transition metal oxide via the ligand group.

In another aspect, there is provided a method of preparing a nanoparticle comprising adding a capping agent to a transitional metal oxide precipitate, the capping agent comprising a ligand group and a functional group, the capping agent coordinating with a transition metal centre in the transition metal oxide precipitate via the ligand group.

In a further aspect, there is provided a method of detecting an analyte molecule in a sample, the method comprising labelling the analyte molecule with a nanoparticle to form a nanoparticle/analyte molecule complex, the nanoparticle comprising a transition metal oxide and a capping agent, the capping agent comprising a ligand group and a functional group, the capping agent coordinated to a transition metal centre in the transition metal oxide via the ligand group, the capping agent reacting with the analyte molecule through the functional amino group; contacting the sample with a working electrode, the working electrode having a surface with a capture molecule disposed thereon to capture the analyte molecule from the sample; contacting the captured analyte molecule that forms the nanoparticle-analyte molecule complex with a redox substrate, under conditions that allow for oxidation or reduction of the redox substrate; and detecting current flow at the working electrode.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures, which illustrate, by way of example only, embodiments of the present invention,

FIG. 1 is a schematic diagram depicting pathways involving microRNA (miRNA);

FIG. 2 is a schematic diagram of an embodiment of a present method for detecting miRNA using electrocatalytic OsO₂ nanoparticles;

FIG. 3 is a TEM image of OsO₂ nanoparticles;

FIG. 4 is a histogram of size distribution of OsO₂ nanoparticles;

FIG. 5 is UV-Vis spectra of (1) 0.25 isoniazid; (2) 0.20 mg/mL uncapped nanoparticles; and (3) 0.20 mg/mL isoniazid capped nanoparticles;

FIG. 6 is cyclic voltammograms of 2.5 mmol/L hydrazine at a capture probe-coated electrode (1) before and (3) after hybridization to complementary let-7b miRNA followed by incubation with nanoparticles; and (2) the hybridized electrode in bland PBS, at a potential scan rate of 25 mV/s;

FIG. 7 is a graph depicting the dependence of the normalized catalytic current at −0.10 V on the hydrazine concentration of (1) 1.0 and (2) 200 pmol/L let-7b hybridized electrodes;

FIG. 8 is a graph depicting the dependence of the catalytic current of 30 mmol/L hydrazine on applied potential of (1) 1.0 and (2) 200 pmol/L let-7b hybridized electrodes (for clarity, the current of (1) was scaled up 50 times);

FIG. 9 depicts the amperometric responses of 5.0 pmol/L (1) let-7b, (2) let-7c and (3) mir-106 hybridized to electrodes complementary to let-7b; and

FIG. 10 is calibration curves for (o) let-7b, ( V̅) mir-106 and (⋄) mir-139 using 30 mmol/L hydrazine and an applied voltage of −0.10 V (insert: calibration curves at low concentration end).

### DETAILED DESCRIPTION

There is presently provided a method of preparing an electrochemically active nanoparticle, which nanoparticle is useful in electrochemical assays to detect analyte molecules in a sample. The nanoparticles are composed of a transition metal oxide and a capping agent, and may be used to amplify an electrochemical detection signal, thus allowing for detection of small quantities of analyte molecule, as well as detection of small analyte molecules that are not easily detected using other methods.

As used herein, the term nanoparticle is intended to refer to a single nanoparticle and to a plurality of nanoparticles, unless otherwise indicated. Thus, reference to a nanoparticle includes reference to one or more nanoparticles, including a dispersion of nanoparticles.

Thus, in one aspect, there is provided a method of preparing a nanoparticle, the method comprising forming a transition metal oxide precipitate from a solution containing a transition metal salt; and adding a capping agent to the transition metal oxide precipitate.

Hydrolysis has been used to synthesize transition metal oxide nanoparticles, which tend to hydrolyze under neutral or alkaline conditions, forming metal hydroxides or oxides.²¹ The present method takes advantage of the fact that nanoparticle nucleation and growth occur via a simple precipitation reaction from homogeneous solution, involving reaction of a metal salt solute with hydroxide or water. To achieve the desired size and size distribution, the growth of the nanoparticles is arrested by addition of a capping agent.

The transition metal salt may be any transition metal salt. As used herein, a transition metal is any metal from the d block of the periodic table. In a particular embodiment, the transition metal salt is a platinum group metal salt. The platinum group metals include ruthenium, rhodium, palladium, osmium, iridium, and platinum. In a further embodiment, the transition metal salt is an osmium salt, and in one particular embodiment is an osmium (IV) salt.

The transition metal salt may comprise one or more alkaline earth metals, one or more halides, and/or one or more ammonium ions, and may be for example, K₂OSCl₆.

To form the precipitate, the transition metal salt may first be dissolved in a suitable solvent. For example, the transition metal salt may be dissolved to a concentration from about 0.1 mg/mL to about 10 mg/mL.

The solvent may be any solvent in which the transition metal salt may be dissolved, but in which the transition metal oxide is not soluble and from which the transition metal oxide can thus be precipitated. Alternatively, the solvent may be a solvent in which the transition metal oxide may be soluble, but to which a further solvent or component may be added to render the transition metal oxide insoluble, thus causing the transition metal oxide to precipitate. For example, the solvent may be a water/ethanol mixture, a water/methanol, a water/acetone or a water/acetonitrile mixture. In one embodiment, the solvent is a water/ethanol mixture with a ratio of 20/80.

In order to form the transition metal oxide precipitate, a hydroxide base is added to the solution of a transition metal salt in an amount sufficient to reduce the a transition metal salt and form the transition metal oxide, for example in a molar ratio of about 0.1/1 of hydroxide/transition metal. In one embodiment, the hydroxide base is sodium hydroxide. In a particular embodiment, sodium hydroxide is added to a final concentration of from about 50 to about 200 μmol/L.

The hydroxide base is added under conditions sufficient to form the transition metal oxide and to allow it to precipitate from solution. For example, the solution containing the transition metal salt and base may be heated, optionally with stirring, for a sufficient time period for the transition metal oxide precipitate to form. For example, the base may be added slowly, such as in a dropwise manner. The solution may then be heated to a temperature of from about 30° C. to about 50° C., or to about 40° C., while stirring, for about 15 minutes to about 1 hour, or for about 30 minutes.

Once the transition metal oxide precipitate is formed, the capping agent is added.

The capping agent is any molecule that is capable of forming a coordination bond with the transition metal ion, thus acting as a ligand for the transition metal, and which has a free functional group available for reaction with a complementary functional group in another molecule, such as an analyte molecule that is to be labelled with the nanoparticle.

The ligand group is any ligand group capable of forming a coordination bond with a transition metal ion, for example, any group in the capping agent that has lone pair electrons or pi electrons available for sharing with the transition metal centre. For example, the ligand group may comprise an aromatic group, a conjugated pi system, a pi bond, a nitrogen atom, an oxygen atom, a sulphur atom or a phosphorus atom. In certain embodiments, the ligand group comprises an aryl group, a diene group, or a triene group.

“Transition metal centre” as used herein refers to the transition metal ion that forms the metal coordination centre for the transition metal oxide, including when the transition metal oxide is complexed with the capping agent, or when oxidized or reduced in a redox reaction. “Osmium centre” or “Os centre” as used herein refers to the Os⁴⁺ ion that forms the metal coordination centre for the OsO₂ complex, including when complexed with the capping agent and/or reduced in a redox reaction to the Os³⁺ ion.

The functional group that is available for reaction with a complementary functional group in another molecule may be any functional group, and is not involved in coordinating with the transition metal centre. In one embodiment, the functional group is a primary amino group, including where the primary amino group forms part of a carbazoyl group (—CONHNH₂), but which is not part of the ligand group and is thus not involved in coordinating with the transition metal centre.

A “functional group” is used herein in its ordinary meaning to refer to an atom or group of atoms within a molecule that impart certain chemical or reactive characteristics to the molecule and which function as a reactive unit within the molecule. It will be understood that complementary functional groups are groups that react with each other to form a bond, including an electrostatic, hydrophobic, hydrogen or covalent bond.

As a result, the capping agent is grafted onto the nanoparticle, meaning that the capping agent is coordinated with a transition metal centre in the nanoparticle via the ligand group in the capping agent, and has a free functional group, such as a primary amino group, available for reaction with a complementary functional group.

Thus, the particular capping agent chosen will influence the nature of the nanoparticle, including charge capacity and capacity retention during electrochemical reactions. A skilled person can readily determine the effect of a given capping agent on the nanoparticle using standard techniques, including those described in the examples that follow.

Addition of the capping agent serves to inhibit growth of the nanoparticles forming in the precipitate, as well as narrowing the size distribution of the nanoparticles. That is, the capping agent may dissolve smaller nanoparticles, leaving a more uniform distribution of nanoparticles in the precipitate. The capping agent also functions to stabilize the nanoparticle, including preventing aggregation of the particles.

Thus, the timing of addition of the capping agent, as well as the amount of capping agent added will affect the size of the final nanoparticles. In certain embodiments, the capping agent is added to a molar ratio of capping agent/transition metal centre of about 10/1.

In a particular embodiment, the capping agent is isoniazid. In another particular embodiment, the capping agent is isoniazid added to a final concentration of about 10 mmol/L.

The capping agent is incubated with the transition metal oxide precipitate for a time sufficient to allow the capping agent to coordinate with a transition metal centre in the transition metal oxide precipitate. For example, the capping agent may be incubated with the transition metal oxide precipitate for about 5 minutes to about 1 hour, or for about 30 minutes.

The resulting nanoparticle may be spherical in shape, having a diameter of from about 1 nm to about 100 nm, from about 2 nm to about 100 nm, from about 5 nm to about 50 nm, or from about 20 nm to about 30 nm.

Once the nanoparticle is formed, the nanoparticle may be washed to remove unreacted reagents. The wash solution should be a solvent or solution in which the nanoparticle is not soluble. For example, the nanoparticle may be washed with ethanol to remove excess transition metal salt and/or capping agent.

The nanoparticle may be removed from the solvent using standard methods, for example filtration or evaporation of the solvent to yield the nanoparticle.

The capping agent acts as a doping agent to interrupt the growth of the nanoparticle as it is forming, and thus controls the size and size distribution of the nanoparticles. The capping agent also serves to stabilize the nanoparticle, in part preventing aggregation.

Also contemplated in another aspect is a nanoparticle, comprising a transition metal oxide and a capping agent, the capping agent including a group that functions as a ligand for coordinating with an osmium centre and a functional group, as described above. In a particular embodiment, the nanoparticle comprises OsO₂ as the transition metal oxide.

Due to inclusion of the transition metal centres in the nanoparticle, the nanoparticle is electroactive, and can be used as an electrocatalyst in an electrochemical detection assay. Where the transition metal oxide is OsO₂, the nanoparticle has a redox potential of approximately −300 to 300 mV relative to a Ag/AgCl electrode.

As well, due to inclusion of the capping agent in the nanoparticle, a desired analyte molecule can be directly or indirectly labelled with the nanoparticle, thus allowing for specific detection of the desired analyte molecule using an electrochemical assay.

That is, through reaction of the functional group in the capping agent, the capping agent is able to react with a complementary functional group in the analyte molecule to be detected, thus forming a bond, including a covalent bond, an electrostatic bond, a hydrogen bond or a hydrophobic bond, between the capping agent and the analyte molecule. The functional group in the analyte molecule may be any functional group that can interact with or react with the complementary functional group in the capping agent.

For example, the capping agent may contain a free primary amino group that is able to react with a carbonyl carbon in the analyte molecule to form a covalent amide bond between the capping agent and the analyte molecule.

Alternatively, the capping agent can be used to indirectly label the analyte molecule by reacting with a functional group in a labelling molecule, the labelling molecule then being able to bind with the analyte molecule.

Thus, there is also presently contemplated an electrochemical assay method for the detection of biological analyte molecules in a sample. The method utilizes the redox active electrocatalytic transition metal oxide moiety of the nanoparticle to amplify an electric signal in the presence of analyte molecule, as well as an interaction between the capping agent and the analyte molecule in order to associate the amplified electrical signal with the analyte molecule.

Accordingly, the method is based on the association of the transition metal oxide complex with the analyte molecule, which allows for detection of the analyte molecule by detecting current generated by a redox reaction catalyzed by the transition metal centre. The transition metal centre catalyzes oxidation or reduction of a redox substrate; electrons are then transferred between the transition metal centre and a working electrode, which is connected through a circuit to a detector that is able to measure current flow. Since the concentration of transition metal oxide-containing nanoparticles is directly proportional to the concentration of the analyte molecule, the present method can be standardized to allow for quantification of the analyte molecule concentration in solution.

The electron exchange between the transition metal centre and the working electrode resets the oxidation state of the transition metal centre, making it available to participate in multiple rounds of the redox reaction and electron transfer, which results in amplification of the signal associated with detection of the analyte molecule. Such a feature of the method enables detection of very small quantities of analyte molecule in a sample.

The amplification feature of the method also makes the method particularly useful for the detection of small oligonucleotides in a sample. Current amplification detection methods such as PCR are not suitable for a short oligonucleotide, since if an oligonucleotide is too short, it cannot act as template for the annealing of primers. The present method allows for detection of short oligonucleotides by capture from a sample and combines amplification of the detection signal so as to allow for detection of very small concentrations of the oligonucleotides. For example, oligonucleotides as short as five nucleotides in length can be detected using the present method, although it will be appreciated that the longer the oligonucleotide, the greater specificity of the method, since there is greater risk of cross-reactivity when identification is based on a short nucleotide sequence.

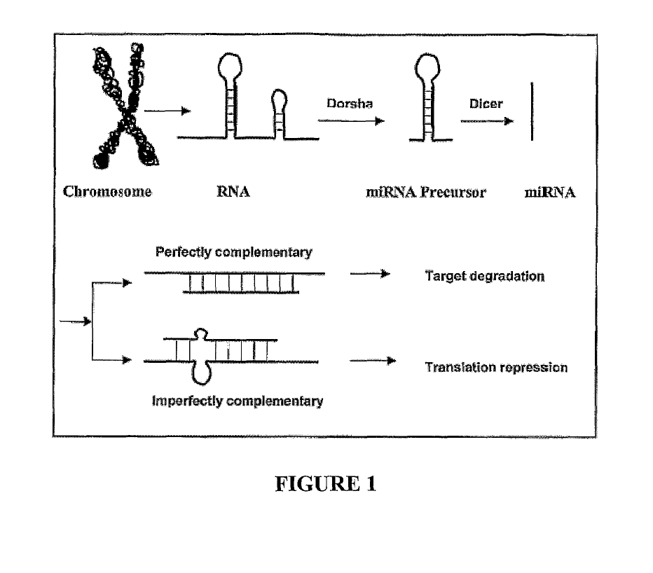

The present method is particularly suited for the detection or quantification of microRNA molecules. MicroRNAs (miRNAs) are a class of 17- to 25-nucleotide (nt) RNA molecules encoded in the genomes of plants and animals that regulate the expression of genes by binding to the 3′-untranslated regions (3′-UTR) of mRNAs. MicroRNAs are transcribed from chromosomes as longer molecules that are processed by a nuclear RNAse, Drosha, to ˜70-nt hairpin miRNA precursors with 3′-overhangs. These precursors are transported to the cytoplasm where they are processed by another RNAse, Dicer, to produce the mature miRNAs (see FIG. 1)^{1,2}.

Recently there has been tremendous interest in this class of small, regulatory RNAs although the first miRNA was reported in the early 90's³. MicroRNAs regulate gene expression through a dual-mechanism, translational repression and target degradation (FIG. 1). In addition to their regulatory roles on gene expression, miRNAs are believed to have great potential in therapeutics, drug discovery, and molecular diagnostics.⁴

A major obstacle in miRNA research is the lack of ultrasensitive miRNA quantification techniques. Therefore, there is an urgent need to develop an accurate and inexpensive assay for miRNA expression analysis. The extremely small size of miRNAs renders most conventional biological amplification tools ineffective because of the inability for much smaller primers/promoters (8- to 10-nt) to bind on such small miRNA templates.^{5,6} For example, RT-PCR can only be used to quantify miRNA precursors rather than the mature miRNAs. Likewise, most of the ultrasensitive two-probe assays (sandwich-type assays), such as gold nanoparticle-based assays⁷ and enzyme-amplified assays^{8,9} have rather limited applications in miRNA analysis, although it has been shown that the sensitivity of those assays is comparable to that of PCR-based fluorescent assays.

Earlier attempts of miRNA expression analysis include Northern blot and cloning. Both techniques have been helpful to spatially and temporally establish the miRNAs expression patterns. ¹⁰ A modified version of Northern blot using locked nucleic acid modified oligonucleotides was developed by Valoczi et al.¹¹ The sensitivity was improved by 10-fold compared to conventional DNA probes.¹¹ As an improvement to Northern blot, the use of nylon macroarrays for miRNA analysis has also been reported.¹² However, Northern blot and cloning techniques suffer from poor sensitivity and involve laborious procedures although Northern blot remains to be the gold standard of miRNA validation and quantitation.¹³

To work with mature miRNAs, various biological ligations have been proposed. For instance, Miska and co-workers proposed an array-based miRNA expression profiling technique, in which miRNAs are ligated to 3′ and 5′ adaptor oligonucleotides followed by RT-PCR.¹⁴ Thomson proposed a T4 RNA ligase procedure to couple the 3′ ends of miRNAs to fluorophore-labeled nucleotides, thereby avoiding the use of RT-PCR.¹⁵ More recently, Nelson presented a procedure called the RNA-primed, array-based Klenow enzyme (RAKE) assay. The RAKE assay uses a Klenow reaction to primer-extend in the 3′ to 5′ direction along the immobilized capture probe only after it hybridized with its complementary miRNA. It has been demonstrated that the assay offers better discrimination against mismatches at the 3′ end, where miRNA homologs share the greatest sequence discrepancy.

In view of the extremely small size of miRNAs, direct chemical ligation of miRNAs themselves may be more advantageous. For example, Babak proposed a cisplatin-based chemical ligation procedure for miRNAs.¹⁶ One binding site of cisplatin is covalently bound to a fluorophore and the other site is a labile nitrate ligand. Incubation in an aqueous solution with miRNAs at elevated temperatures results in a ligand exchange between the labile nitrate of cisplatin and the more strongly coordinating N₇ purine nitrogen of G base, forming a new complex between cisplatin and G base. MicroRNAs are therefore directly labeled with cisplatin-fluorophore conjugates through coordinative bonds with G bases.

Another chemical ligation procedure at the 3′ end was developed by Liang.¹⁷ Incubation with biotinylated hydrazide renders biotin at the 3′ end of miRNAs. After the introduction of quantum dots to the hybridized miRNAs through reacting with quantum dots-avidin conjugates, the miRNAs were detected fluorescently with a dynamic range of 156 pM to 20 nM. Nonetheless, the much needed sensitivity in miRNA assay remains to be realized.

To further enhance the sensitivity and lower the detection limit, the present methods couple a chemical ligation procedure to an electrochemical amplification scheme. The present methods are based a direct chemical ligation procedure that involves a chemical reaction to tag analyte molecules such as miRNAs with the transition metal oxide nanoparticles. The nanoparticles effectively catalyze the oxidation of hydrazine and greatly enhance the detectability of small analyte molecules such as miRNAs, thereby lowering the detection limit to femtomolar levels. In practice, this sensitivity of the assay meets the requirements for direct miRNA expression profiling.

The present method is rapid, ultrasensitive, non-radioactive, and is able to directly detect an analyte molecule. By employing transition metal oxide nanoparticles, an analyte molecule can be directly labeled with redox and electrocatalytic moieties. When applied to detection of specific miRNA, these molecules may be detected amperometrically at subpicomolar levels with high specificity.

Thus, in another aspect, there is provided a method for detecting an analyte molecule in a sample.

The method comprises labelling the sample with a nanoparticle as described herein to form a nanoparticle-analyte molecule complex. The nanoparticle-analyte molecule complex is contacted with a working electrode that has a capture molecule disposed on a surface of the working electrode, thus capturing the nanoparticle-analyte molecule complex.

Alternatively, the analyte molecule may first be captured by the capture molecule and then labelled with the nanoparticle to form the nanoparticle-analyte molecule complex. Thus, although the following description generally relates to labelling of the sample containing the analyte prior to capture of the analyte, the present method also contemplates adaptation to allow for capture of an unlabelled analyte molecule from the sample followed by labelling of the captured analyte with the nanoparticle.

Thus, in one embodiment, a redox substrate is contacted with the captured nanoparticle-analyte molecule complex under conditions that allow for oxidation or reduction of the redox substrate. Current flow is then detected at the working electrode, which is in circuit with a counter electrode, a biasing source and a device for measuring current flow.

The sample is any sample in which an analyte molecule is desired to be detected, and may comprise a biological sample including a biological fluid, a tissue culture or tissue culture supernatant, a prepared biochemical sample including a prepped nucleic acid sample such as a prepped RNA sample or including a prepped protein sample, a field sample, a cell lysate or a fraction of a cell lysate.

The analyte molecule may be any analyte molecule that is desired to be detected in a sample and which is capable of labelling, either directly or indirectly, with a nanoparticle as described herein. If the analyte molecule is to be labelled directly, it will contain a functional group that is accessible for reaction with or binding to the capping agent, such that reaction with or binding to the capping agent does not interfere with capture of the analyte molecule by the capture molecule.

In various embodiments, the analyte molecule comprises a protein, a peptide, DNA, RNA including mRNA and microRNA, or a small molecule. The analyte molecule should be stable enough under the labelling conditions so as to allow for detection once complexed with the nanoparticle. In certain embodiments, the analyte molecule comprises a microRNA, for example the let-7b microRNA.

In one embodiment, the analyte molecule is an RNA molecule comprising the sequence UGAGGUAGUAGGUUGUGUGGUU [SEQ ID NO: 1]. In another embodiment, the analyte molecule is an RNA molecule consisting essentially of the sequence of SEQ ID NO: 1. In another embodiment, the analyte molecule is an RNA molecule consisting of the sequence of SEQ ID NO: 1.

“Consisting essentially of” or “consists essentially of” as used herein means that a molecule may have additional features or elements beyond those described provided that such additional features or elements do not materially affect the ability of the molecule to function as an analyte molecule or a capture molecule, as the case may be. That is, the molecule may have additional features or elements that do not interfere with the binding interaction between analyte and capture molecule. For example, a peptide or protein consisting essentially of a specified sequence may contain one, two, three, four, five or more additional amino acids, at one or both ends of the sequence provided that the additional amino acids do not block, interrupt or interfere with the binding between the peptide or protein and its target molecule, either analyte or capture molecule. In a further example, a nucleic acid molecule consisting essentially of a specified nucleotide sequence may contain one, two, three, four, five or more nucleotides at one or both ends of the specified sequence provided the nucleic acid molecule can still recognize and bind to its target analyte or capture molecule. Similarly, a peptide, protein or nucleic acid molecule may be chemically modified with one or more functional groups provided that such chemical groups do not interfere with the interaction between the analyte molecule and the capture molecule to prevent or reduce the ability of the capture molecule to bind the analyte molecule.

It will be appreciated that the analyte molecule should be stable enough under conditions for labelling to allow for recognition and capture by the capture molecule. For example, if the analyte molecule comprises a protein that is to be labelled directly, it should be stable enough under labelling conditions to maintain any structural features that may be required for capture of the analyte molecule by the capture molecule.

As well, it will be appreciated that where the analyte molecule comprises a double stranded nucleic acid, the sample should be heated to a sufficient temperature to melt the double stranded nucleic acid prior to labelling, so as to allow for subsequent capture by a capture molecule having a sequence that is complementary to at least a portion of one strand of the double stranded nucleic acid.

The analyte molecule may be labelled directly with the nanoparticle, without need for isolation of the analyte molecule from the sample. The analyte molecule is reacted with the nanoparticle under conditions suitable to allow for the functional group in the capping agent to react with a complementary functional group in the analyte molecule.

For example, where the analyte molecule is a nucleic acid, the analyte molecule may be treated with a strong reducing agent, for example sodium periodate, to reduce the 3′ sugar residue to a di-aldehyde, which is then available for reaction with a group such as a free amino group in the capping agent.

Thus, when being labelled directly, the sample containing the analyte molecule, which possesses one or more functional groups available for reaction with the capping agent, is contacted with the nanoparticle, resulting in formation of a nanoparticle/analyte molecule complex. The nanoparticle/analyte molecule complex may be formed through covalent, electrostatic or hydrogen bonds, for example.

Alternatively, the analyte molecule may be labelled indirectly by use of a labelling molecule. The labelling molecule will contain one or more functional groups available for reaction with the nanoparticle so that it can form a bond with the nanoparticle in the same manner as described above for an analyte molecule that contains a suitable functional group.

As well, the labelling molecule will recognize and bind the analyte molecule within the sample, having greater affinity for the analyte molecule than for other molecules that may be present in the sample. It will be appreciated that the labelling molecule should bind to the analyte molecule in such a way so as not to interfere with capture of the analyte molecule by the capture molecule disposed on the working electrode.

The labelling molecule may comprise a protein, a peptide, a ligand, an antibody, a nucleic acid binding protein or protein domain, or an oligonucleotide, or a small molecule, including biotin and digoxin, containing an available functional group.

If the sample volume is large enough, the nanoparticle may be added directly to the sample. Alternatively, the labelling may be done in a suitable buffer in which both the nanoparticle and the analyte molecule are stable, by mixing of the nanoparticle and the sample in a suitable labelling buffer. In exemplary embodiments, the buffer may contain a salt at a concentration from about 1 mM to about 2 M and may have a pH from about 4 to about 11. The precise buffer chosen will depend in part on the nature of the sample and the nature of the analyte and/or capture molecule.

If the analyte molecule or labelling molecule contains more than one suitable functional group, not every available functional group will necessarily be labelled with the nanoparticle. The density of labelling which results will depend in part on the distribution and arrangement of the functional groups in the molecule to be labelled.

As stated above, it will be appreciated that labelling of the analyte molecule directly, or indirectly through use of a labelling molecule, may be done prior to capture of the analyte molecule or following capture of the analyte molecule. Depending on the nature of the capture molecule and functional groups contained in the capture molecule, as well as the desired reaction between the analyte molecule and the capping agent, it may be desirous to label the analyte molecule or labelling molecule prior to capture, so as not to result in labelling of capture molecules, which would give an inflated electrochemical signal in the present method, increasing the background signal of the method. Alternatively, if labelling of the analyte molecule prior to capture is liable to interfere with the interaction between the analyte molecule and the capture molecule, it may be desirous to first capture the analyte molecule as described above, prior to labelling with the nanoparticle.

The sample containing the analyte molecule is contacted with a working electrode on which a capture molecule is disposed.

The capture molecule is a molecule that recognizes and specifically binds to the analyte molecule. “Specifically binds” or “specific binding” means that the capture molecule binds in a reversible and measurable fashion to the analyte molecule and generally has a higher affinity for the analyte molecule than for other molecules in the sample. The capture molecule should recognize and bind to the analyte molecule even when the analyte molecule has been labelled, either directly or indirectly, to form a nanoparticle/analyte molecule complex. However, as mentioned above, labelling of the analyte molecule may be done following capture of the analyte molecule by the capture molecule.

The capture molecule may comprise a protein, a peptide, a nucleic acid including DNA, RNA and an oligonucleotide, a ligand, a receptor, an antibody or a small molecule.

In one embodiment, the capture molecule is a single stranded oligonucleotide with a complementary sequence to the sequence of a single stranded nucleic acid analyte molecule. In one embodiment, the capture molecule is a single stranded oligonucleotide with a sequence complementary to that of a microRNA that is to be detected in the sample.

In a particular embodiment, the capture molecule is a single stranded oligonucleotide comprising a sequence that is complementary to the sequence of the let-7b microRNA. In one embodiment, the capture molecule comprises the sequence AACCACACAACCTACTACCTCA [SEQ ID NO: 2]. In another embodiment, the capture molecule consists essentially of the sequence of SEQ ID NO: 2. In another embodiment, the capture molecule consists of the sequence of SEQ ID NO: 2.

The capture molecule is disposed on a surface of the working electrode, meaning that the capture molecule is coated on, immobilized on, or otherwise applied to the working electrode surface. The disposition may involve an electrostatic, hydrophobic, covalent or other chemical or physical interaction between the capture molecule and the working electrode surface. For example, the capture molecule may be chemically coupled to the electrode. Alternatively, the capture molecule may form a monolayer on the surface of the electrode, for example through self-assembly mechanisms.

The capture molecule should be disposed on the working electrode surface at a density such that the capture molecule can readily recognize and bind the analyte molecule. For example, if the capture molecule is an oligonucleotide, the capture molecule may be disposed on the working electrode surface at a density of about 6.0×10⁻¹² mol/cm² or greater, of about 8.5×10⁻¹² mol/cm² or less, or from about 6.0×10⁻¹² mol/cm² to about 8.5×10⁻¹² mol/cm².

The term “working electrode” refers to the electrode on which the capture molecule is disposed, and means that this electrode is the electrode involved in electron transfer with the transition metal centre during the redox reaction. The working electrode may be composed of any electrically conducting material, including carbon paste, carbon fiber, graphite, glassy carbon, any metal commonly used as an electrode such as gold, silver, copper, platinum or palladium, a metal oxide such as indium tin oxide, or a conductive polymeric material, for example poly(3,4-ethylenedioxythiophene) (PEDOT) or polyaniline. In a particular embodiment, the working electrode is indium tin oxide.

The sample is contacted with the capture molecule on the surface of the working electrode under conditions and for a time sufficient for the capture molecule to recognize and bind the analyte molecule. For example, if the capture molecule is a single stranded oligonucleotide for capturing a single stranded nucleic acid from solution, the sample is added to the working electrode surface along with a suitable hybridization buffer, and the sample is incubated with the capture molecule for sufficient time under mild to stringent hybridization conditions to allow for recognition and binding of the analyte microRNA molecule by the complementary oligonucleotide capture molecule.

For example, the sample may be incubated with the capture probe at a temperature of about 30° C. for about 60 minutes, in a hybridization buffer containing phosphate buffered-saline (pH 8.0), consisting of 0.15 M NaCl and 20 mM NaCl.

Once the nanoparticle/analyte molecule complex has been captured by the capture molecule at the surface of the working electrode (or alternatively, once the captured analyte molecule has been labelled with the nanoparticle to form the nanoparticle/analyte molecule complex), the working electrode may optionally be rinsed to remove excess sample or hybridization buffer, for example, 3 to 5 times with a suitable buffer. The rinsing buffer should be of an appropriate pH and buffer and salt concentration so as not to interfere with or disrupt the interaction between the capture molecule and analyte molecule.

After the nanoparticle/analyte molecule complex has been captured by the capture molecule, a redox substrate is added to the working electrode surface in a buffer and under conditions suitable for oxidation or reduction of the redox substrate by the transition metal centre.

The redox substrate is a molecule that is capable of being oxidized or reduced by the transition metal centre. If the redox substrate is to be oxidized by the transition metal centre, it will have a redox potential that is less positive than the transition metal centre; similarly, when the redox substrate is to be reduced by the transition metal centre, it will have a redox potential that is more positive than the transition metal centre.

Thus, the redox substrate may be any molecule that can be oxidized or reduced by the transition metal centre in a redox reaction. In a particular embodiment, the redox substrate is hydrazine. In another particular embodiment, the redox substrate is ascorbic acid.

As will be appreciated, the working electrode will form part of an electrochemical cell. An electrochemical cell typically includes a working electrode and a counter electrode. In the case of a two-electrode system, the counter electrode functions as a reference electrode. In a three-electrode system the electrochemical cell further comprises a separate reference electrode.

In various embodiments the reference electrode may be a Ag/AgCl electrode, a hydrogen electrode, a calomel electrode, a mercury/mercury oxide electrode or a mercury/mercury sulfate electrode.

The electrodes within the electrochemical cell are connected in a circuit to a biasing source, which provides the potential to the system. As well, a device for measuring current, such as an ammeter, is connected in line. The electrodes are in contact with a solution that contains a supporting electrolyte for neutralization of charge build up in the solution at each of electrodes, as well as the redox substrate that is to be oxidized or reduced. In order to initiate the redox reaction, a potential difference is applied by the biasing source. A current can flow between counter electrode and the working electrode, which is measured relative to the reference electrode.

Typically, the applied potential difference is at least 50 mV more positive than the redox potential of the transition metal centre or at least 50 mV more negative than redox potential of the transition metal centre, depending on the analyte is being oxidized or reduced.

The current generated as a result of electron transfer catalysed by the transition metal centre will be directly proportional to the concentration of the transition metal centre, and therefore to the concentration of the captured analyte molecule, allowing for quantification of the concentration of the analyte molecule. The current that flows at the working electrode is derived from transition metal centres that are specifically associated with captured analyte molecules.

A skilled person will understand how to perform a standard curve with known concentrations of a particular analyte molecule, and as described in the Examples set out herein, so as to correlate the level of detected current with detection of a given concentration of the analyte molecule. In this way, the present method can be used to quantify levels of an analyte molecule in a sample.

Since the redox substrate, for example hydrazine, is in excess in the present method, once a particular transition metal centre has been reduced or oxidized through an interaction with a redox substrate molecule, the transition metal centre can be oxidized or reduced by electron exchange with the electrode, resetting the transition metal centre and making it available for a subsequent round of redox reaction with another redox substrate molecule.

For example, the mechanism of oxidation of the redox substrate hydrazine by OsO₂ is represented by the following equations:

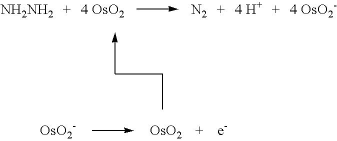

Thus, the present method is sensitive and is able to detect very small quantities of analyte molecule in a sample. For example, for detecting microRNAs in a sample, the present method may have a detection range of about 0.20 to about 300 pM, with a lower detection limit of about 80 fM in a 2.5 μl volume. This means that as little as about 0.2 attomole of microRNA may be detected using the present method, and that as little as about 5 ng of total RNA preparation may be required as a sample to detect microRNAs.

For each of the above steps, the appropriate solution may be added to the surface of the working electrode using a liquid cell, which may be a flow cell, as is known in the art, or by pipetting directly onto surface of the working electrode, either manually or using an automated system. The liquid cell can form either a flow through liquid cell or a stand-still liquid cell.

Due to electrode technology that allows for miniaturization of electrodes, the above method can be designed to be carried out in small volumes, for example, in as little as 1 μl volumes. In combination with the very low detection limit, this makes the present method a highly sensitive method of detecting an analyte molecule in a sample, which is applicable for use in point-of-care and in-field applications, including disease diagnosis and treatment, environmental monitoring, forensic applications and molecular biological research applications.

The present methods are well suited for high throughput processing and easy handling of a large number of samples. This electrochemical miRNA assay is easily extendable to a low-density array format of 50-100 working electrodes. The advantages of low-density electrochemical biosensor arrays include: (i) more cost-effective than optical biosensor arrays; (ii) ultrasensitive when coupled with electrocatalysis; (iii) rapid, direct, while being turbidity- and light absorbing-tolerant and (iv) portable, robust, low-cost, and easy-to-handle electrical components suitable for field tests and homecare use.

Thus, to assist in high volume processing of samples, the working electrode may be used in an array of electrodes. Multiple working electrodes may be formed in an array, for use in high throughput detection methods as described above. Each working electrode in the array may comprise a different capture molecule, for detecting a number of different analyte molecules simultaneously. Alternatively, each working electrode in the array may comprise the same capture molecule, for use in screening a number of different samples for the same analyte molecule.

Each working electrode may be located within a discrete compartment, for ease of applying the same or different sample to each surface of each working electrode. Alternatively, each working electrode can be arrayed so as to contact a single bulk solution. An automated system can be used to apply and remove fluids and sample to each working electrode.

A different capture molecule for detecting a particular analyte molecule within a sample may be disposed on respective working electrodes. Each working electrode may then be contacted with the same sample so as to detect multiple analyte molecules within a single sample at one time.

Alternatively, multiple working electrodes may be arranged in an array such that each individual working electrode has the same capture molecule disposed on its surface. A different sample may then be contacted with each respective working electrode. In this way a large number of samples may be screened for a particular analyte molecule.

### EXAMPLES

Materials: K₂OsCl₆ (>99%), isoniazid (99%), sodium periodate (99%), sodium borohydride (>99%), 3-aminopropyl trimethoxysilane (97%), and mono-n-dodecyl phosphate (MDP) were purchased from Sigma-Aldrich (St Louis, Mo.). ITO coated glass slides were from Delta Technologies Ltd (Stillwater, Minn.). Three human miRNAs, let-7b (22 nt), mir-106 (24 nt), and mir-139 (18 nt),¹⁸ were selected as our target m1RNAs. Aldehyde-modified oligonucleotide capture probes used in this work were custom-made by Invitrogen Corporation (Carlsbad, Calif.) and all other oligonucleotides of PCR purity were from 1st Base Pte Ltd (Singapore). Conducting epoxy was purchased from Ladd Research (Williston, Vt.). All other reagents were obtained from Sigma-Aldrich and used without further purification. A pH 6.0 0.20 mol/L sodium acetate buffer containing 2.0 mmol/L sodium periodate was used as the hybridization buffer. To minimize the effect of RNases on the stability of miRNAs, all solutions were treated with diethyl pyrocarbonate and surfaces were decontaminated with RNaseZap™ (Ambion, Tex.).

Apparatus: Electrochemical experiments were carried out using a CHI 660A electrochemical workstation coupled with a low current module (CH Instruments, Austin, Tex.). The working electrode was a 2.0-mm-diameter ITO electrode. Electrical contact was made to the ITO electrode using the conducting epoxy and a copper wire. The contact formed had a resistance<1.0Ω. All electrochemical measurements were performed using a three-electrode system consisted of the ITO working electrode, a miniature Ag/AgCl reference electrode (Cypress Systems, Lawrence, Kans.), and a platinum wire counter electrode. A pH 8.0 phosphate-buffered saline (PBS) was used as the supporting electrolyte. UV-Vis spectra were recorded on a V-570 UV/VIS/NIR spectrophotometer (JASCO Corp., Japan). X-ray photoelectron spectroscopic (XPS) data were collected on a VG ESCALAB 220I-XL XPS system (Thermo VG Scientific Ltd., UK). Scanning electron microscopic (SEM) and transmission electron microscopic (IBM) tests were conducted on a JSM-7400F electron microscope (Joel Ltd., Tokyo, Japan).

Preparation of the OsO₂ nanoparticles: The OsO₂ nanoparticles were prepared in a water/ethanol (20/80) mixture solvent containing NaOH. For a typical preparation, NaOH dissolved in water/ethanol (50/50) was slowly added to a solution of K₂OsCl₆ in 100 ml of the water/ethanol (20/80) mixture solvent. The final concentration of NaOH was between 50 and 200 μmol/L. Several minutes after mixing the precursors, the mixture was then heated to 40° C. for ˜30 min to produce the nanoparticles. Isoniazid, dissolved in the mixture solvent, was added to the nanoparticle solution to a final concentration of 10 mmol/L. After another 30 min of stirring, 100 ml of ethanol was added and the mixture was centrifuged at 10,000 rpm. The nanoparticles were then washed with ethanol several times.

Electrode fabrication: Prior to capture probe immobilization, an ITO slide was silanized following a published procedure.¹⁹ A patterned 2-mm thick adhesive spacing/insulating layer was assembled on the top of the slide, forming a low-density electrode array of 20-30 2-mm-diameter individual electrodes. 5.0 L aliquots of 0.50 μmol/L aldehyde-modified capture probes in pH 6.0, 0.10 moL/L acetate buffer were applied to the individual electrodes and incubated for 3 h at room temperature in a moisture-saturated environmental chamber. After incubation, the electrodes were rinsed successively with 0.10% SDS and water. The reduction of imine was carried out by a 5 minute incubation of the electrodes in 2.5 mg/mL sodium borohydride solution made of PBS/ethanol (3/1). The electrodes were then soaked in vigorously stirred hot water (90-95° C.) for 2 min, copiously rinsed with water, and blown dry with a stream of nitrogen. To improve the quality and stability of the electrodes, and minimize non-hybridization-related nanoparticle uptake, the capture probe-coated electrodes were immersed in 2.0 mg/mL MDP for 3-5 h. Unreacted MDP molecules were rinsed off and the electrodes were washed by immersion in a stirred ethanol for 10 min, followed by a thorough rinsing with water. The surface density of the immobilized capture probes was found to be in the range of 5.0-8.0 pmol/cm².²⁰

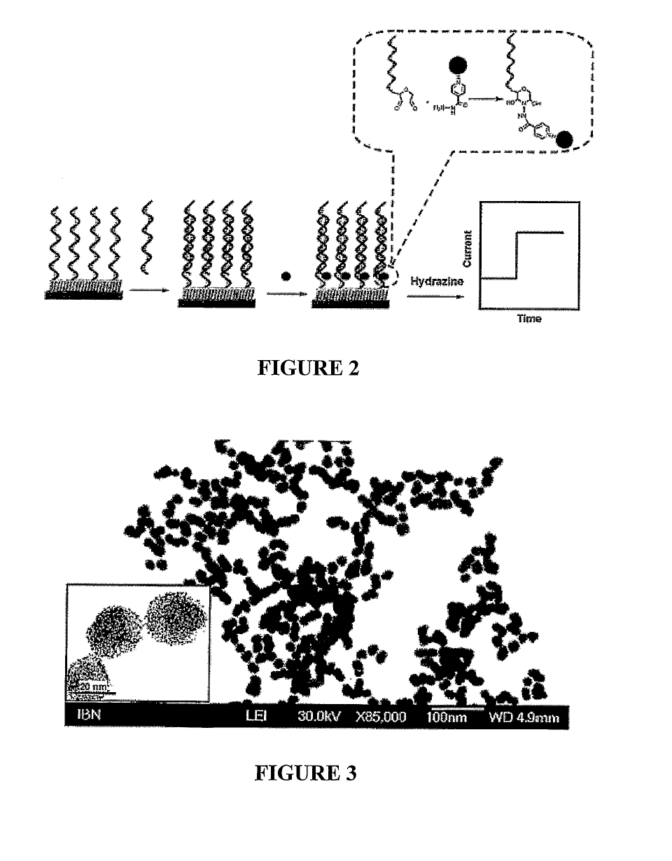

Total RNA extraction, derivation, hybridization, and detection: Total RNA from human HeLa-60 cells was extracted using TRIzol™ reagent (Invitrogen, Carlsbad, Calif.) according to the manufacturer's recommended protocol. MicroRNAs in the total RNA were enriched using an YM-50 Montage spin column (Millipore Corp., Billerica, Mass.). RNA concentration was determined by UV-Vis spectrophotometry. The hybridization and nanoparticles tagging of miRNA and its amperometric detection were carried out in three steps, as depicted in FIG. 2. First, the electrodes were placed in the environmental chamber. 2.0 μL aliquots of the total RNA solution in pH 6.0 0.20 mol/L acetate buffer were placed on the electrodes. 0.50 μL Aliquots of 10 mmol/L sodium periodate in the acetate buffer were added on the electrodes and mixed thoroughly with the total RNA solution.

The hybridization cum derivation of the 3′ overhangs of the miRNAs was carried out at 25° C. in the dark for 60 min. After a thorough washing with 0.10 mmol/L sodium sulfite in the acetate buffer, 5.0 μL aliquots of 0.10 mg/mL the nanoparticles in the acetate buffer were then added and the electrodes were incubated at 30° C. for 4 h. After another thorough washing with the acetate buffer, the electrodes were characterized electrochemically.

Finally, amperometric detection of the miRNAs was performed on the electrode array at −0.10 V in 30 mmol/L hydrazine in PBS. The individual electrode remained open-circuit until a 10 μL aliquot of the PBS test solution was applied. Withdrawal of the test solution effectively disabled the electrode. In the case of lower miRNA concentrations, smoothing was applied after each measurement to remove random noises. All potentials reported in this work were referred to the Ag/AgCl electrode and sill experiments were carried out at room temperature, unless otherwise stated.

### RESULTS AND DISCUSSION

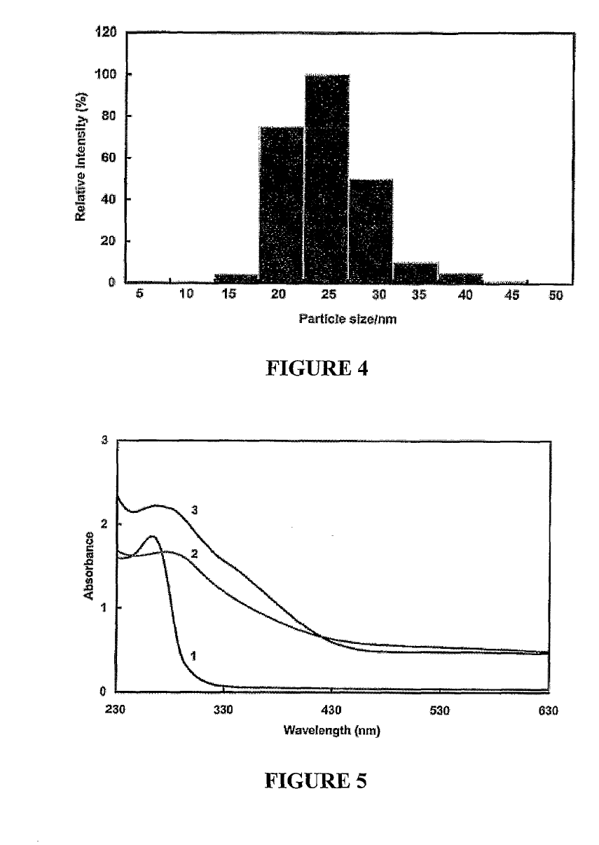

Formation of the OsO₂ nanoparticles: OSO₂ nanoparticles in the range of 5.0 to 50 nm were prepared through modulating the reaction conditions. The nanoparticles were first characterized by TEM, as it provides a direct visualization of the quality of the nanoparticles, i.e. their shape, size, and size distribution. A typical TEM image and a size-distribution histogram of the nanoparticles are shown in FIGS. 3 and 4. It is seen from FIG. 3 that the nanoparticles are approximately spherical and mono-dispersed. Particle size distribution analysis revealed that most of the particles are from 20-30 nm with a mean diameter of 25 nm (FIG. 4). The excellent particle size distribution may be explained by enrichment of larger nanoparticles during isoniazid capping. The capping, resulting in some loss of the nanoparticles, significantly narrows the particle size distribution by eliminating (dissolving) smaller ones.

FIG. 5 shows the UV-Vis absorption spectra of the nanoparticles before and after capping. The spectrum of the nanoparticles before capping is more or less characteristic of the spectra for nanoparticles: a rather broad absorption band stretches over 200 nm (FIG. 5 trace 2).²¹ The spectrum of the capped nanoparticles appeared as a superposition of the isoniazid (FIG. 5 trace 1) and the uncapped nanoparticles (FIG. 5 trace 2) with an additional shoulder in the 330-430 nm region (FIG. 5 trace 3), indicating that the capping agent is successfully grafted onto the nanoparticles.

To assign the oxidation state and stoichiometry of the nanoparticles, we used XPS to study the nanoparticles before and after capping. As listed in Table 1, the Os_{4f} doublet OS_{4f5/2} and OS_{4f7/2}, Os_{5p3/2}, and ₁ₛ were observed in the nanoparticles before capping, which agrees well with that of OsO₂ within the experimental errors.²² A characteristic N₁ₛ was observed after capping, suggesting the presence of isoniazid on the nanoparticles. The O/Os and N/Os ratios, calculated from the integrated XPS high-resolution bands after cross-section correction, were 2.3 and 1.60, respectively. The presence of significant amount of N indicates that multiple isoniazid molecules are grafted on the nanoparticles, providing anchoring sites for miRNA.

TABLE 1
X-ray Photoemission Spectroscopy data of the nanoparticles

| | | Os | | O | N |
|---|---|---|---|---|---|
| | 4f_{7/2} | 4f_{5/2} | 5p_{3/2} | 1ₛ | 1ₛ |
| OsO₂ nanoparticles | 51.6 | 54.4 | 45.7 | 530.4 | — |
| (uncapped) | | | | | |
| OsO₂ nanoparticles | 51.7 | 54.5 | 45.6 | 530.4 | 400.2 |
| (capped) | | | | | |
| OsO₂^{a} | 51.7 | 54.5 | 45.8 | 530.2 | — |
| Element/Os ratio (capped) | — | — | — | 2.3 ± 0.60 | 1.6 ± 0.40 |

^{a}Data from Ref. 22.

Application of the nanoparticles in ultrasensitive miRNA assay: Nucleic acid assays with electrocatalytic tags have previously been reported.^{23,24} The tags chemically amplify analytical signals to hybridized electrodes compared to non-hybridized ones. The differences in amperometric currents are used for quantification purpose. In a similar way, the nanoparticles were evaluated as the electrocatalytic tags for in the present ultrasensitive miRNA assay.

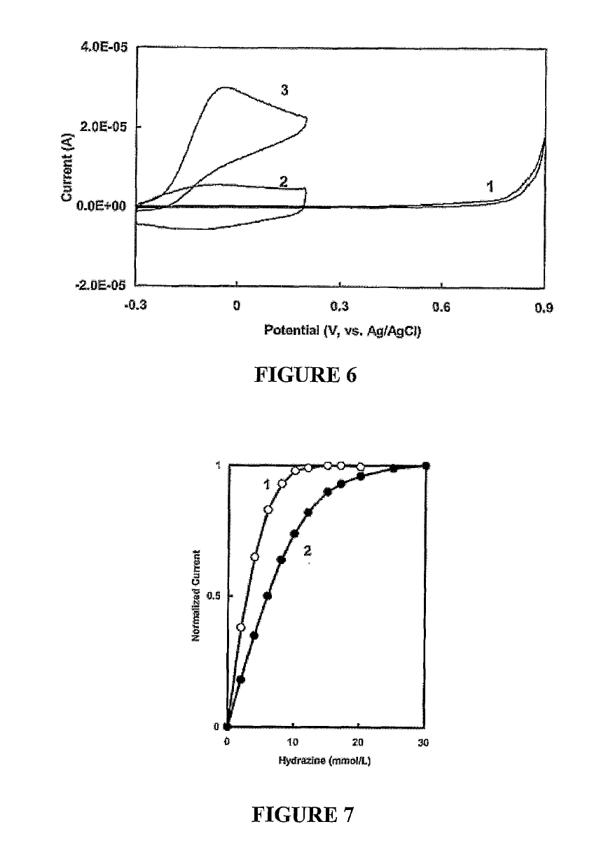

FIG. 6 shows cyclic voltammograms of the electrodes in PBS containing hydrazine after hybridization with mir-106 (noncomplementary, control) and let-7b (complementary, analyzed miRNA), and after incubation with the nanoparticles. Upon hybridization, let-7b was selectively captured and bond to the electrode, where little if any of mir-106 was captured during hybridization. Incubation of the hybridized electrode with the nanoparticles grafts the nanoparticles onto the hybridized miRNA molecules through a condensation reaction between isoniazid and the 3′ end dialdhydes of miRNA.²⁵ For comparison, a voltammogram of the hybridized electrode in blank PBS is also presented (FIG. 6, trace 2).

As expected, the voltammograms of the control electrode before and after mir-106 treatment were indistinguishable (FIG. 6, trace 1). Moreover, little current for the oxidation of hydrazine at potentials<0.80 V was observed at the control electrode, as expected with the slow heterogeneous electron-transfer rate of hydrazine, caused by a high oxidation overpotential at the ITO electrode. It is well documented that direct oxidation of hydrazine suffers from very high overpotentials. Reported values for its oxidation range from 0.30-1.0 V.^{26,27,28} The presence of the mixed monolayer on the electrode further impedes the electron-transfer. On the other hand, a pair of very broad current peaks of the hybridized and nanoparticles treated electrode were observed at −0.10 V, which increased with the concentration of let-7b (FIG. 6, trace 2). It is apparent that the nanoparticles exhibit an improvement in response for the oxidation hydrazine: the oxidation of hydrazine appeared at −0.10 V, essentially the same potential as that of the nanoparticles themselves. There was a significant improvement in the sharpness of the current peak. The current was enhanced by a factor of ˜10³ compared with that at the control electrode at the same potential, and the cathodic current of the nanoparticles was suppressed to an extent that was close to zero at higher hydrazine concentrations (FIG. 6, trace 3).

These results suggest that there is a strong catalytic effect by the nanoparticles, since the current at potentials in the vicinity of the nanoparticles redox potential increased dramatically and the overpotential of hydrazine oxidation was reduced by as much as 900 mV, indicating that the nanoparticles are being turned over by the oxidation of hydrazine. The increase in peak current and the decrease in the oxidation overpotential demonstrate an efficient electrocatalysis of hydrazine. The shift in the overpotential is due to a kinetic effect and hence greatly increases the electron transfer rate from hydrazine to the electrode.

The catalytic current was found to be pH dependent, and the maximum value was obtained in the pH range of 8.0-9.0. Therefore, subsequent experiments were performed at pH 8.0. It was found that similar catalytic effect is observed at a gold electrode. The electrocatalytic oxidation potential of hydrazine by the nanoparticles at the gold electrode was practically identical to that of the ITO electrode. However, the overpotential of hydrazine oxidation at the gold electrode was much lower, 0.30-0.40 V. A considerable background current was obtained at potentials where miRNA quantification was conducted, making the gold electrode less favourable.

Controlled-potential electrolysis at 0.20 V revealed that the number of electrons involved in the catalytic oxidation of hydrazine is ˜4.^{26,27,28} Therefore, the mechanism of the oxidation of hydrazine at the hybridized electrode may be presented by the following equations:

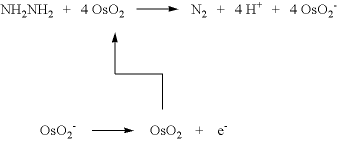

Because the ITO electrode is inactive to hydrazine at potentials<0.80 V, the nanoparticles immobilized on the ITO electrode act as nanoelectrodes for the oxidation of hydrazine, forming a nanoelectrode array. Moreover, at the hydrazine oxidation potential, the thus reduced nanoparticles are instantly oxidized, generating a substrate-recycling mechanism, as described by the above equations. These results demonstrate that miRNA selectively hybridizes with its complementary capture probe on the electrode surface with very little cross-hybridization; the nanoparticle tags are successfully ligated on the hybridized miRNA molecules; and the nanoparticles effectively catalyze the oxidation of hydrazine, producing a much enhanced analytical signal.

Andrieux and co-workers have analyzed in great details the electrocatalytic process, taking into consideration of all possible steps involved.³¹ In the case of the catalytic oxidation of hydrazine by the nanoparticles, the rate determining step(s) is likely to be one of the following: (i) mass-transport process of hydrazine in solution, (ii) catalytic process at the nanoparticle-solution interface, and (iii) electron-transfer at the electrode-nanoparticle interface. Under extreme circumstances, when both the catalytic process and the mass-transport in solution are much faster than the electron transfer at the nanoparticle-electrode interface, the limiting current is then solely controlled by the electron-transfer process, which in turn, by the total number of nanoparticles at the electrode surface, thereby by the concentration of the analyzed miRNA in solution. This sole electron-transfer-controlled process can be achieved by “speeding up” mass-transport and “slowing down” electron-transfer rate because little can be done in modulating the catalytic process. The mass-transport rate is directly proportional to the concentration of the substrate. High mass-transport rates are obtained when working with high concentrations of substrate.

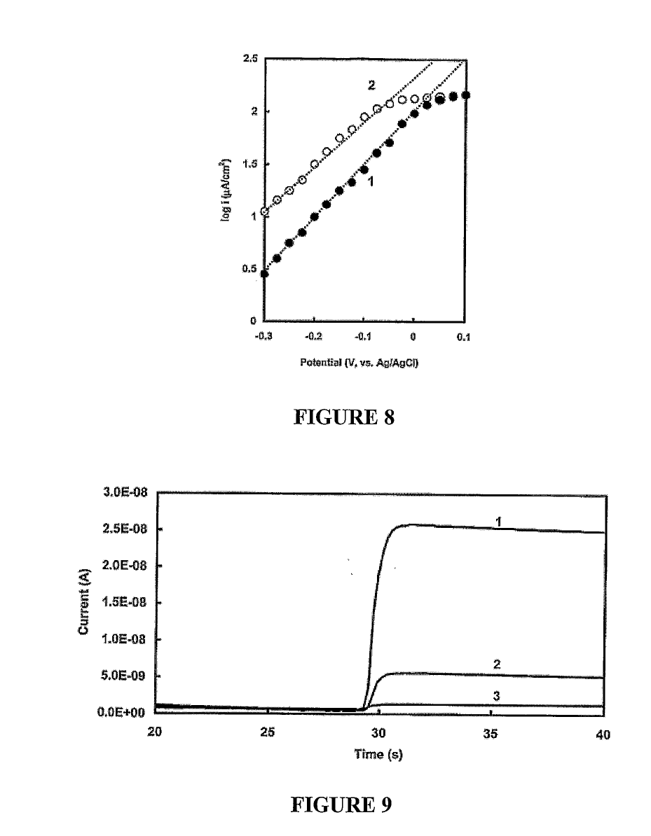

As shown in FIG. 7, the catalytic current was practically independent of hydrazine concentration at ≧30 mmol/L, implying that the catalytic current is now controlled by the electron-transfer process. Meanwhile, a low electron-transfer rate is achievable by increasing the electron hopping distance between the nanoparticle and the electrode because the electron-transfer rate decreases exponentially with increasing the thickness of the insulating monolayer on the electrode. It was found that the blocking treatment with MDP after the immobilization of the capture probes effectively slows down the electron-transfer rate. Moreover, the electron-transfer rate is also dependent on the applied potential, E, according to the Bulter-Volmer equation,³² which provides a much more convenient means for manipulating the electron transfer rate. As illustrated in FIG. 8, the linear segments of the log i vs. E plots indicate that the overall process is solely controlled by the electron-transfer at the nanoparticle-electrode interface. The deviations from linearity at higher applied potentials come from limitations imposed by mass-transport, as the electron-transfer is accelerated to such an extent that mass-transport is becoming the rate-limiting step.

The above experiments suggest that a linear relationship between the current arid the analyzed miRNA exists under conditions of high hydrazine concentrations (≧30 mmol/L) and low applied potentials (<−0.050 V). Therefore, all subsequent amperometric measurements were conducted in 30 mmol/L hydrazine at −0.10 V.

As demonstrated in FIG. 9, upon addition of 30 mmol/L hydrazine to PBS, the oxidation current in amperometry increased to 26 nA at the electrode hybridized to 5.0 pmol/L of the complementary let-7b (FIG. 9 trace 1), whereas in the control experiment that used the non-complementary mir-106, only a 0.90 nA increment in hydrazine oxidation current was observed (FIG. 9 trace 3), largely due to the residual non-hybridization-related uptake of the nanoparticles.

The specificity of the assay for the detection of target miRNA was further evaluated by analyzing let-7b and let-7c with electrodes coated with the capture probes complementary to let-7b. There is only one nucleotide difference (G [symbol] A) out of 22 nucleotides of between let-7b and let-7c, meaning that the capture probe for let-7b is one base-mismatched for let-7c. As shown in trace 2 in FIG. 9, the current increment dropped by ˜80% to as low as 5.0 nA when let-7c was tested on the electrode, readily allowing discrimination between the perfectly matched and mismatched miRNAs. It was found that the nanoparticles with a diameter of 5.0 to 25 nm produce the most sensitive signal and their optimal concentrations are from 0.050 to 0.20 mg/mL. The amperometric data agree well with the voltammetric results obtained earlier and confirm that the target miRNA is successfully detected with high specificity and sensitivity. Therefore, each quantified result represents the specific quantity of a single miRNA member and not the combined quantity of the entire family.

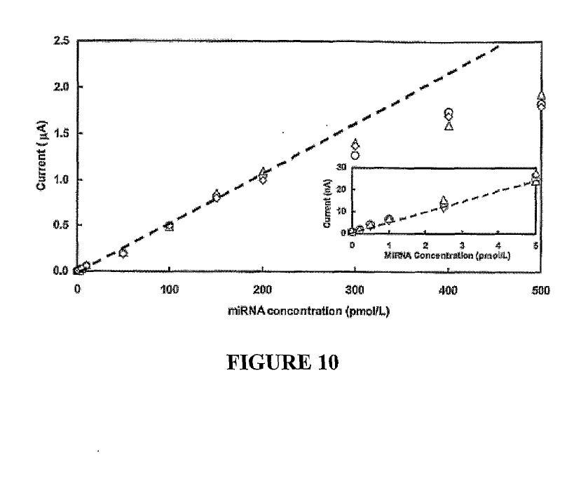

Calibration curves for miRNAs: In this study, three representative miRNAs of 18 to 24 nucleotides were selected. For the control experiment, capture probes non-complementary to any of the three miRNAs were used in the electrode preparation. As illustrated in FIG. 10, the dynamic range was 0.30-200 pmol/L with a detection limit of 80 fmol/L. Compared to previous chemical ligation-based miRNA assays, the sensitivity of the assay is increased by combining the direct chemical ligation with an amplification scheme.

In this assay the ratio of the nanoparticle tag to target miRNA molecule is fixed at 1. The amount of the capture probes immobilized on the electrode surface and hybridization efficiency determine the amount of target miRNA bound to the electrode and thereby the amount of the nanoparticles. At the same molar concentration, the sensitivity should be independent of the size of miRNAs. Indeed, as shown in FIG. 10, a practically constant sensitivity for all three miRNAs was obtained irrespective to their lengths.

Analysis of miRNA Extracted from HeLa 60 cells: The assay was applied to the analysis of the three miRNAs in total RNA extracted from HeLa-60 cells, to determine its ability in quantifying miRNAs in real world samples. The results were normalized to total RNA, as listed in Table 2.

TABLE 2
Analysis of miRNAs in total RNA extracted from HeLa 60 cells

| | Let-7b | Mir-106 | Mir-139 |
|---|---|---|---|
| | (copy/μg RNA) | (copy/μg RNA) | (copy/μg RNA) |
| This method | 5.2 ± 0.68 × 10⁷ | 2.7 ± 0.43 × 10⁷ | 0.23 ± 0.029 × 10⁷ |
| Northern | 5.5 ± 0.66 × 10⁷ | 2.4 ± 0.41 × 10⁷ | 0.25 ± 0.032 × 10⁷ |
| blot | | | |

These results are in good agreement with those obtained by Northern blot assay on the same sample and consistent with recently published data of miRNA expression profiling.^{33,34,35} The lowest amount of total RNA needed for a successful miRNA detection was found to be ˜5.0 ng, corresponding to ˜150 HeLa cells.^{34,36} The relative errors associated with the assay were generally less than 15% in the concentration range of 1.0 to 200 pmol/L. Therefore, the assay is capable of identifying miRNAs with less than 2-fold difference in expression levels under two conditions. This is advantageous because the expressions of many of the most interesting miRNAs often differ slightly under different conditions.

The present assay offers accuracy in the identification of differentially expressed miRNAs and cuts down on the need for running too many replicates. With the improved sensitivity, the assay also significantly reduces the amount of total RNA needed from micrograms to nanograms.

As can be understood by one skilled in the art, many modifications to the exemplary embodiments described herein are possible. The invention, rather, is intended to encompass all such modification within its scope, as defined by the claims.

All documents referred to herein are fully incorporated by reference.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. All technical and scientific terms used herein have the same meaning as commonly understood by one of ordinary skill in the art of this invention, unless defined otherwise.

### REFERENCES

(1) Lee, Y.; Ahm, C.; Han, J.; Choi, H.; Kim, J.; Yim, J.; Lee, J.; Provost, P.; Radmark, O.; Kim, S. Nature 2003, 425, 415-419
(2) Hutvagner, G.; Zamore, P. D. Science 2001, 293, 834-838
(3) Lee, R. C.; Feinbaum, R. L.; Ambros, V. Cell 1993, 75, 843-854
(4) Lu, J.; Getz, G.; Miska, E. A.; Alvarez-Saavedra, E.; Lamb, J.; Peck, D.; Sweet-Cordero, A.; Ebert, B. L; Mak, R. H.; Ferrando, A. A.; Downing, J. R.; Jacks, T.; Horvitz, H. R.; Golub, T. R. Nature 2005, 435, 834-838
(5) Liu, C. G.; Calm, G. A.; Meloon, B.; Gamliel, N.; Sevignani, C.; Ferracin, M.; Dumitru, C. D.; Shimizu, M.; Zupo, S. Proc. Natl Acad. Sci. USA, 2004, 101, 9740-9744
(6) Calm, G. A.; Liu, C. G.; Sevignani, C.; Ferracin, M.; Felli, N.; Dumitru, C. D.; Shimizu, M.; Cinimino, A. Proc. Natl Acad. Sci. USA, 2004, 101, 11755-11760
(7) Rosi, N. L.; Mirkin, C. A. Chem. Rev. 2005, 105, 1547-1562
(8) Zhang, Y.; Kim, H. H.; Heller, A. Anal. Che i. 2003, 75, 3267-3269
(9) Xie, H.; Zhang, C.; Gao, Z. Anal Chem. 2004, 76, 1611-1617
(10) Bartel, D. P. Cell 2004, 116, 281-297
(11) Valoczi, A.; Hornyik, C.; Varga, N.; Burgyan, J.; Kauppinen, S.; Havelda, Z. Nucleic Acid Res. 2004, 32, e175
(12) Krichevsky, A. M.; King, K. S.; Donahue, C. P.; Khrapko, K.; Kosik, K. S. RNA 2003, 9 1274-1281
(13) Ambros, V.; Bartel, B.; Bartel, D. P.; Burge, C.; Carrington, J.; Chen, X.; Dreyfuss, G.; Eddy, R.; Griffiths-Jones, S.; Marshall, M.; Matzke, M.; Ruvlun, G.; Tuschl, T. RNA 2003, 9, 277-279
(14) Miska, E. A.; Alvarez-Saavedra, E.; Townsend, M.; Yoshii, A.; Sestan, N.; Rakic, P.; Constantine-Paton, M.; Horvitz, H. R. Genome Biol 2004, 5, R68
(15) Thomson, J. M.; Parker, J.; Perou, C. M.; Hammond, S. M. Nature Methods, 2004, 1, 47-53
(16) Babak, T.; Zhang, W.; Morris, Q.; Blencowe, B. J.; Hughes, T. R. RNA, 2004, 10, 1813-1819
(17) Liang, R. Q.; Li, W.; Li, Y.; Tan, C.; Li, J.; Jin, Y.; Ruan. K. C. Nucleic Acids Res. 2005, 33, e17
(18) Griffiths-Jones S. Nucleic Acids Res. 2004, 32, D109-D111
(19) Hedges, D. H. P.; Richardson, D. J.; Russell, D. A. Langmuir 2004, 20, 1901
(20) Gao, Z.; Tansil, N. C. Nucleic Acid Res. 2005, 33, e123
(21) Murray, C. B.; Kagan, C. R.; Bawendi. M. G. Annu. Rev. Mater. Sci. 2000, 30,
(22) Sarma, D. D.; Rao, C. N. R. J. Electron. Spectrosc. Relat. Phenomn. 1980, 20,
(23) Tansil, N. C.; Xie, H.; Xie, F.; Gao, Z. Anal. Chem. 2005, 77, 126-134
(24) Tansil, N. C.; Xie, H.; Gao, Z. Chem. Commun. 2005, 1064-1066
(25) Hansske, F.; Cramer, F. Carbohyd. Res. 1977, 54, 75-84
(26) Ozoernena, K. I.; Nyokong, T. Talanta 2005, 67, 162-168
(27) Pournaghi-Azer, M. H., Sabzi, R. J. Electroanal. Chem. 2003, 543, 115-125
(28) Hou, W.; Ji, H.; Wang, E. Talanta 1992, 39, 45-50
(29) Morf, W. E. Anal Chem. Acta 1996, 330, 139-149
(30) Saito, Y. Rev. Plarogr. 1968, 15, 177-189
(31) Andrieux, C. P.; Dumas-Boucbiat, J. M.; Saveant, J. M. J. Electroanal. Chem. 1982, 131, 1-20
(32) Bard A. J.; Faulkner, L. R. Electrochemical Methods, John Wiley & Sons, New York, 2001; p 100
(33) Barad, O.; Meiri, E.; Avniel, A.; Aharonov, R.; Barsilai, A.; Bentwich, I.; Einav, U.; Gilad, S.; Hurhan, P.; Karov, Y.; Lobenhofer, E. K.; Sharon, E.; Shiboletli. Y. M.; Shtutman, M.; Bentwich, Z.; Einay, P. Genome Res. 2004, 14, 2486-2494
(34) Allawi, H. T.; Dahiberg, J. E.; Olson, S.; Lund, E.; Olson, M.; Ma, W. P.; Takova, T.; Neri, B. P.; Lyamichev, V. I. RNA, 2004, 10, 1153-1161
(35) Nelson, P. T.; Baldwin, D. A.; Scearce, L. M.; Oberholtzer, J. C.; Tobias, J. W.; Mourelatos. Z. Nature Methods, 2004, 1, 155-161
(36) Lim, Li'.; Lau, N. C.; Weinstein, E. G.; Abdelhakim, A.; Yekta, S.; Roades, M. W.; Burge, C. B.; Bartel, D. P. Gene & Dev. 2003, 17, 991-1008

1. A nanoparticle comprising a transition metal oxide and a capping agent, the capping agent comprising a ligand group and a functional group, the capping agent coordinated to a transition metal centre in the transition metal oxide via the ligand group, the functional group being available for reaction with an analyte molecule.

2. The nanoparticle of claim 1 wherein the transition metal oxide is a platinum group metal oxide.

3. The nanoparticle of claim 1 wherein the transition metal oxide is OsO₂.

4. The nanoparticle of claim 1 wherein the functional group is a primary amino group.

5. The nanoparticle of claim 1 wherein the ligand group is an aryl group.

6. The nanoparticle of claim 5 wherein the capping agent is isoniazid.

7. The nanoparticle of claim 1 having a diameter of from about 5 to about 50 nm.

8. The nanoparticle of claim 7 having a diameter of from about 20 to about 30 nm.

9. A method of preparing a nanoparticle of claim 1 comprising:
adding a capping agent to a transition metal oxide precipitate, the capping agent comprising a ligand group and a functional group, the capping agent coordinating with a transition metal centre in the transition metal oxide precipitate via the ligand group, wherein the functional group is available for reaction with an analyte molecule.

10. The method of claim 9 wherein the transition metal oxide is a platinum group metal oxide.

11. The method of claim 9 wherein the transition metal precipitate is formed by adding a hydroxide base to a solution of a transition metal salt.

12. The method of claim 11 wherein the transition metal salt comprises one or more alkaline earth metals, one or more halides or an ammonium ion.

13. The method of claim 9 wherein the transition metal oxide is OsO₂.

14. The method of claim 11 wherein the transition metal salt is K₂OsCl₆.

15. The method of claim 11 wherein the solution comprises 20/80 ratio of water/ethanol.

16. The method of claim 15 wherein the hydroxide base is sodium hydroxide.

17. The method of claim 9 wherein the functional group is a primary amino group.

18. The method of claim 9 wherein the ligand group is an aryl group.

19. The method of claim 17 wherein the capping agent is isoniazid.

20. A method of detecting an analyte molecule in a sample, the method comprising:
labelling the analyte molecule with a nanoparticle of claim 1 to form a nanoparticle/analyte molecule complex, the capping agent reacting with the analyte molecule through the functional group;
contacting the sample with a working electrode, the working electrode having a surface with a capture molecule disposed thereon to capture the analyte molecule from the sample;
contacting the captured analyte molecule that forms the nanoparticle-analyte molecule complex with a redox substrate, under conditions that allow for oxidation or reduction of the redox substrate; and
detecting current flow at the working electrode.

21. The method of claim 20 wherein the labelling occurs prior to contacting the sample with the working electrode.

22. The method of claim 20 wherein the labelling occurs after contacting the sample with the working electrode.

23. The method of claim 20 wherein the transition metal oxide is a platinum group metal oxide.

24. The method of claim 20 wherein the transition metal oxide is OsO₂.

25. The method of claim 20 wherein the functional group is a primary amino group.

26. The method of claim 20 wherein the ligand group is an aryl group.

27. The method of claim 25 wherein the capping agent is isoniazid.

28. The method of claim 20 further comprising rinsing the working electrode prior to contacting the redox substrate with the captured analyte molecule.

29. The method of claim 20 wherein the sample comprises a biological sample, a tissue culture, a tissue culture supernatant, a prepared biochemical sample, a field sample, a cell lysate or a fraction of a cell lysate.

30. The method of claim 29 wherein the biological sample comprises a biological fluid and the prepared biochemical sample comprises a prepped nucleic acid sample or a prepped protein sample.

31. The method of claim 30 wherein the sample comprises a prepped RNA sample.

32. The method of claim 20 wherein the analyte molecule comprises a protein, a peptide, DNA, mRNA, microRNA or a small molecule.

33. The method of claim 20 wherein the analyte molecule is a microRNA.

34. The method of claim 20 wherein the capture molecule comprises a protein, a peptide, DNA, RNA, an oligonucleotide, a ligand, a receptor, an antibody or a small molecule.

35. The method of claim 34 wherein the capture molecule comprises an oligonucleotide having a sequence complementary to the sequence of a microRNA.

36. The method of claim 20 wherein the redox substrate is hydrazine or ascorbic acid.

37. The method of claim 20 wherein the working electrode comprises carbon paste, carbon fiber, graphite, glassy carbon, gold, silver, copper, platinum, palladium, a metal oxide or a conductive polymer.

38. The method of claim 37 wherein the metal oxide is indium tin oxide and the conductive polymer is poly(3,4-ethylenedioxythiophene) (PEDOT) or polyaniline.

39. The method of claim 20 wherein the analyte molecule is labelled directly with the nanoparticle.

40. The method of claim 20 wherein a labelling molecule is used to label the analyte molecule indirectly with the nanoparticle.

41. The method of claim 40 wherein the labelling molecule comprises a protein, a peptide, a ligand, an antibody, a nucleic acid binding protein or protein domain or an oligonucleotide.


SEQUENCE LISTING (2 sequences)

SEQ ID NO: 1 - RNA, 22 nt, Homo sapiens
  human let-7b miRNA
ugagguaguagguugugugguu

SEQ ID NO: 2 - DNA, 22 bp, artificial
  synthetic DNA complementary to sequence of let-7b miRNA
aaccacacaacctactacctca